(19) United States
(12) Patent Application Publication
(10) Pub. No.: US 2023/0404799 A1
(43) Pub. Date: Dec. 21, 2023

(54) METHODS OF DELIVERING THERAPEUTIC AGENTS TO THE OPTIC NERVE HEAD AND DEVICES FOR PRACTICING SAME

(71) Applicant: The Board of Trustees of the Leland Stanford Junior University, Stanford, CA (US)

(72) Inventor: Bryce Chiang, Redwood City, CA (US)

(21) Appl. No.: 18/205,128

(22) Filed: Jun. 2, 2023

Related U.S. Application Data
(60) Provisional application No. 63/348,162, filed on Jun. 2, 2022.

(51) Int. Cl. A61F 9/00; A61F 9/007; A61M 25/00

(52) U.S. Cl. CPC A61F 9/0017; A61F 9/00736; A61M 25/0084; A61M 25/0026; A61M 2202/07; A61M 2210/0612; A61M 2025/0091

(57) ABSTRACT

Provided are methods of delivering a therapeutic agent to the optic nerve head (ONH) of a subject. The methods comprise creating a sclerostomy posterior to the limbus of the subject and feeding a catheter with a distal needle tip tangentially into the sclerostomy. The methods further comprise tunneling the catheter through the suprachoroidal space (SCS) toward the ONH, and disposing the needle tip adjacent, or into, the ONH. The methods further comprise injecting the therapeutic agent adjacent or into the ONH. The methods find use in a variety of contexts, including but not limited to, treating an ONH-associated condition in a subject in need thereof. Examples of such conditions include, but are not limited to, optic neuropathies such as glaucoma, non-arteritic anterior ischemic optic neuropathy (NAION), and the like. Devices and kits that find use in practicing the methods of the present disclosure are also provided.

### CROSS-REFERENCE TO RELATED APPLICATIONS

This application claims the benefit of U.S. Provisional Patent Application No. 63/348,162, filed Jun. 2, 2022, which application is incorporated herein by reference in its entirety.

### STATEMENT REGARDING FEDERALLY SPONSORED RESEARCH OR DEVELOPMENT

This invention was made with Government support under contract EY033407 awarded by the National Institutes of Health. The Government has certain rights in the invention.

### INTRODUCTION

Optic neuropathies, such as glaucomatous optic neuropathy, ischemic optic neuropathies, autoimmune disease, optic disc drusen, and toxic/metabolic optic neuropathies, are vision threatening disorders affecting millions of Americans. Though some optic neuropathies have good visual prognosis in the near term, most cause permanent and sometimes even catastrophic vision loss. This is because as retinal ganglion cell (RGC) axons are lost, they do not spontaneously regenerate, and at some later point, the RGCs die and are not replaced. Thus, new clinical strategies to slow disease progression or promote RGC survival and/or axon growth are needed.

Targeted delivery of therapeutics may be useful in promoting organized axon regeneration because it can (i) reach difficult-to-reach diseased tissues, and (ii) establish spatial cues. In some diseases, such as glaucoma and non-arteritic anterior ischemic optic neuropathy (NAION), the optic nerve head (ONH) is the likely site of injury and targeting the ONH could be used to counteract injury signals that dampen axon regeneration. However, current approaches have failed to demonstrate reliable access to the ONH. Intravitreal injections, intraorbital optic nerve injections, and systemic administration are the current state of the art in dosing the ONH. Intravitreal injections in small animals have demonstrated uptake by the retinal nerve fiber layer. However, a concentration gradient, if it is even established, directed toward the vitreous is unlikely to be physiologic and is not expected to optimally promote axon regeneration down the optic nerve. Furthermore, intravitreal injections deliver drugs to the retinal ganglion cell soma, and less to the RGC axons in the ONH. Intraorbital optic nerve injections have also been used to target the optic nerve, but this is expected to localize drugs behind the lamina cribrosa and it is unclear how such signaling would reach axons or promote regeneration.

### SUMMARY

Provided are methods of delivering a therapeutic agent to the optic nerve head (ONH) of a subject. The methods comprise creating a sclerostomy posterior to the limbus of the subject and feeding a catheter with a distal needle tip tangentially into the sclerostomy. The methods further comprise tunneling the catheter through the suprachoroidal space (SCS) toward the ONH, and disposing the needle tip adjacent, or into, the ONH. The methods further comprise injecting the therapeutic agent adjacent or into the ONH. The methods find use in a variety of contexts, including but not limited to, treating an ONH-associated condition in a subject in need thereof. Examples of such conditions include, but are not limited to, optic neuropathies such as glaucoma, non-arteritic anterior ischemic optic neuropathy (NAION), and the like. Devices and kits that find use in practicing the methods of the present disclosure are also provided.

### BRIEF DESCRIPTION OF THE FIGURES

FIG. 1: Photograph of the optic nerve labeled to show alternative therapeutic agent delivery approaches including: (1) optic nerve injection; (2) oblique optic nerve injection; (3) intravitreal access to the optic nerve; and (4) the approach according to the present disclosure involving suprachoroidal access to the optic nerve.

FIG. 2A-2E: Schematic illustration of a method for delivering therapeutic agents to the optic nerve head according to embodiments of the present disclosure (sometimes referred to herein as SupraChoroidal-to-Optic-NErve (SCONE) injection). (A) Diagram of eye prior to procedure. Sc-sclera, Ch-choroid, Re-retina, VH-vitreous humor, ON-optic nerve, ONH-optic nerve head. (B) A microneedle is used to inject viscoelastic into suprachoroidal space (SCS). (C) A catheter is introduced into the SCS and directed toward the ONH. The method may either proceed with (D) injection into the peripapillary SCS, or (E) needle exposed and penetrated into ONH, followed by injection into ONH.

FIG. 3A-3G: Representative images of enucleated albino rabbit eye snap frozen after injection with India ink. (A) Intraorbital optic nerve injection, (B) Intraorbital optic nerve injection with microneedle, (C) Intraorbital optic nerve injection angled toward optic nerve head, (D) Intravitreal injection, (E) Intravitreal approach to optic nerve head, (F) Suprachoroidal injection, (G) Suprachoroidal tunneling to optic nerve head. Circle indicates 1mm radius centered on optic nerve head. C=cornea, L=lens, ON=optic nerve stalk.

FIG. 4A-4F: Histologic photos of calcium stained with Alizarin Red delivered with (A) intraorbital optic nerve injection, (B) intraorbital optic nerve injection with a microneedle, (C) intraorbital optic nerve injection angled toward optic nerve head, (D) intravitreal injection, (E) intravitreal approach to optic nerve, and (F) suprachoroidal tunneling to the optic nerve. Arrow indicates needle track if present on slide. VH-vitreous humor, Sc-sclera, ON-optic nerve, Ch-choroid.

FIG. 5: Data showing the proportion of successful localization to optic nerve head (ONH) for each injection technique. (A) Intraorbital optic nerve injection, or ION; (B) Intraorbital optic nerve injection with microneedle, or ION with MN; (C) Intraorbital optic nerve injection angled toward optic nerve head, or ION angled; (D) Intravitreal injection, or IVT; (E) Intravitreal approach to optic nerve head, or IVT approach to ONH; (F) Suprachoroidal injection, or SCS injection; (G) Suprachoroidal tunneling to optic nerve head, or SCS tunneling to ONH. * indicates significance (p<0.05) based on binomial distribution compared with 50% chance.

FIG. 6: Summary of results for the effects of hollow microneedle length and hollow microneedle insertion angle for the creation of a sclerostomy. Each box represents one trial.

FIG. 7: Fundus photos demonstrating successful delivery of India ink to the ONH of a rabbit via SCONE injection. “Pre”=pre-injection; POW1, POW3 and POW4 are post-operative weeks 1, 3 and 4, respectively.

FIG. 8: Histochemical staining demonstrating successful delivery of India ink to the ONH of a rabbit via SCONE injection. The asterisk indicates the ONH. India ink is indicated by the arrow.

FIG. 9A-9C: Data demonstrating retention of a functional connectivity between the eye and brain after SCONE injection of India ink to the rabbit eye.

FIG. 10A-10C: Illustrations of a device according to some embodiments of the present disclosure.

FIG. 11: Photos of components of a device according to some embodiments of the present disclosure.

FIG. 12: Illustrations of catheter types that may be employed in the methods and devices of the present disclosure.

### DETAILED DESCRIPTION

Before the methods, devices and kits of the present disclosure are described in greater detail, it is to be understood that the methods, devices and kits are not limited to particular embodiments described, as such may, of course, vary. It is also to be understood that the terminology used herein is for the purpose of describing particular embodiments only, and is not intended to be limiting, since the scope of the methods, devices and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, devices and kits. The upper and lower limits of these smaller ranges may independently be included in the smaller ranges and are also encompassed within the methods, devices and kits, subject to any specifically excluded limit in the stated range. Where the stated range includes one or both of the limits, ranges excluding either or both of those included limits are also included in the methods, devices and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, devices and kits belong. Although any methods, devices and kits similar or equivalent to those described herein can also be used in the practice or testing of the methods, devices and kits, representative illustrative methods, devices and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, devices and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, devices and kits, which are, for clarity, described in the context of separate embodiments, may also be provided in combination in a single embodiment. Conversely, various features of the methods, devices and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, devices and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Delivering Therapeutic Agents to the ONH

Aspects of the present disclosure include methods of delivering therapeutic agents to the optic nerve head (ONH) of a subject. In some embodiments, the methods comprise creating a sclerostomy posterior to the limbus of the subject, feeding a catheter with a distal needle tip tangentially into the sclerostomy, and tunneling the catheter through the suprachoroidal space (SCS) toward the ONH. Such methods further comprise disposing the needle tip adjacent, or into, the ONH, and injecting the therapeutic agent adjacent or into the ONH. The methods find use in treating ONH-associated conditions, non-limiting examples of which include optic neuropathies.

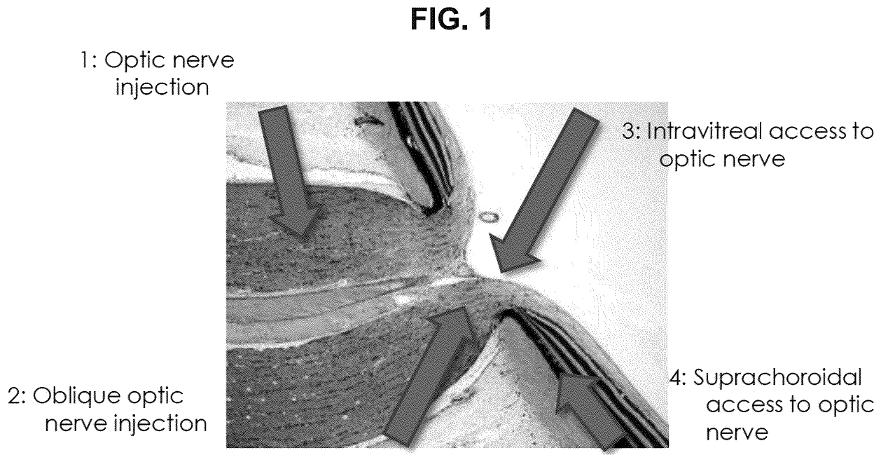

There are currently no satisfactory or FDA-approved approaches for delivering therapeutic agents to the ONH. Existing approaches are illustrated in FIG. 1 and include optic nerve injection, oblique optic nerve injection, and intravitreal access to the optic nerve. Shortcomings of these approaches include one or more of difficulty to visualize intraoperative, potential damage to the lamina cribrosa, and/or potential damage to the ONH itself. The methods of the present disclosure address these shortcomings and provide additional benefits as well. Details regarding the methods are now provided below.

A schematic illustration of a method for delivering therapeutic agents to the optic nerve head according to embodiments of the present disclosure (sometimes referred to herein as SupraChoroidal-to-Optic-NErve (SCONE) injection) is shown in FIG. 1. (A) Diagram of eye prior to procedure. Sc-sclera, Ch-choroid, Re-retina, VH-vitreous humor, ON-optic nerve, ONH-optic nerve head. In this example and as shown in panel (B), a sclerostomy is created using a hollow microneedle followed by injection of a viscoelastic agent into the suprachoroidal space (SCS). Next, in this embodiment and shown in panel (C), a catheter with needle tip, or a sheath with internal catheter and distal needle tip, is introduced into the SCS and tunneled toward the ONH. The method may either proceed with (Di) injection of the therapeutic agent into the peripapillary SCS, or (Dii) needle exposed and penetrated into the ONH, followed by injection of the therapeutic agent into ONH.

The sclerostomy may be created at any suitable location. In certain embodiments, the sclerostomy is created from 0.5 to 7 mm posterior to the limbus, e.g., from 1 to 6 mm, 2 to 5 mm, or 3 to 4 mm posterior to the limbus.

In some instances, the sclerostomy is created by inserting a hollow microneedle through the sclera. The hollow microneedle may be made of any suitable material. According to some embodiments, the hollow microneedle is made of a metal, e.g., stainless steel (e.g., an alloy of iron with carbon, silicon, manganese and chromium). In certain embodiments, the hollow microneedle is made of ceramic, tungsten carbide, titanium, polyetheretherketone (PEEK), nitinol, or any combination thereof.

The dimensions of the hollow microneedle may vary. According to some embodiments, the length of the hollow microneedle is from 300 μm to 1.7 mm, from 400 μm to 1.6 mm, from 500 μm to 1.5 mm, from 750 μm to 1.4 mm, from 800 μm to 1.2 mm, or from 900 μm to 1.1 mm, e.g., about 1 mm. In some instances, the inner diameter of the hollow microneedle is from 100 to 500 μm, from 200 to 400 μm, or from 250 to 350 μm, e.g., about 300 μm.

In certain embodiments, for sclerostomy creation, the microneedle insertion angle (that is, the angle relative to the surface of the sclera at which the microneedle is inserted through the sclera) is 45 to 65 degrees, such as 48 to 62 degrees, 50 to 60 degrees, or from 52 to 58 degrees, e.g., about 55 to 57 degrees.

According to some embodiments, the hollow microneedle is mounted on a distal face (e.g., of a base plate) of a device used to perform the method, and wherein the angle of the microneedle is 45 to 65 degrees, such as 48 to 62 degrees, 50 to 60 degrees, or from 52 to 58 degrees, e.g., about 55 to 57 degrees, relative to the face of the base plate such that the microneedle inserts through the sclera at the angle.

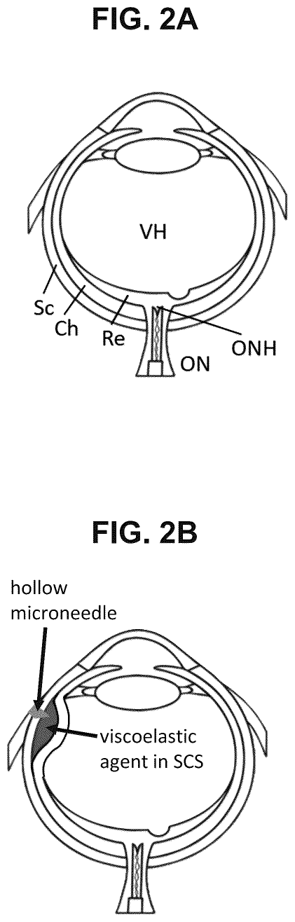

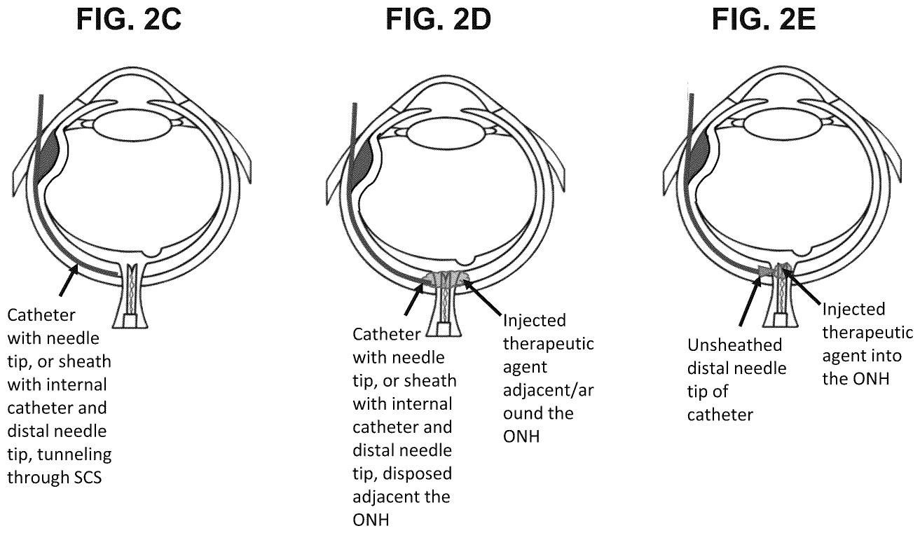

In certain embodiments, when a hollow microneedle is employed to create the sclerostomy, the feeding comprises passing the catheter through the lumen of the hollow microneedle. In some instances, when a hollow microneedle is employed to create the sclerostomy, a viscoelastic agent is injected into the SCS while the hollow microneedle is still inserted through the sclera. See, e.g., FIG. 2B. A viscoelastic material possesses both viscous and elastic characteristics under loading. According to some embodiments, the viscoelastic agent comprises or consists of sodium hyaluronate, chondroitin sulphate, hydroxypropyl methylcellulose (HPMC), or any combination thereof.

The methods of the present disclosure utilize a catheter. The dimensions of the catheter may vary. In certain embodiments, the outer diameter of the catheter is from 100 to 400 μm, e.g., from 150 to 350 μm, from 200 to 300 μm, or from 225 to 275 μm. The inner diameter of the catheter may similarly vary and in some instances is from 50 to 250 μm, from 75 to 225 μm, or from 100 to 200 μm, e.g., from 125 to 175 μm.

The catheter may be made of any suitable material. In certain embodiments, the catheter is made of plastic. According to some embodiments, the catheter is made of silicone, polyurethane (PU), polyethylene (PE), polyvinylchloride (PVC), PTFE, latex, Teflon, rubber (e.g., red rubber), nylon, or any combination thereof. According to some embodiments, the catheter is made of a metal, e.g., stainless steel (e.g., an alloy of iron with carbon, silicon, manganese and chromium). In certain embodiments, the needle tip is made of ceramic, tungsten carbide, titanium, polyetheretherketone (PEEK), nitinol, or any combination thereof.

According to some embodiments, the feeding comprises feeding the catheter tangential to the sclera at an angle of from 20 to 40 degrees, or from 25 to 35 degrees, e.g., about 30 degrees.

In some instances, the catheter comprises a needle tip and the needle tip is disposed adjacent, or into, the ONH. In other embodiments, the catheter does not comprise a needle tip. For example, in some embodiments, when the therapeutic agent is to be injected adjacent (rather than into) the ONH, the catheter may or may not comprise a distal needle tip.

When the catheter comprises a distal needle tip, the needle tip bevel angle is from 25 to degrees, from 35 to 50 degrees. The needle tip may be made of any suitable material. According to some embodiments, the needle tip is made of a metal, e.g., stainless steel (e.g., an alloy of iron with carbon, silicon, manganese and chromium). In certain embodiments, the needle tip is made of ceramic, tungsten carbide, titanium, polyetheretherketone (PEEK), nitinol, or any combination thereof.

In certain embodiments, during the feeding and tunneling steps, the catheter (e.g., including a distal needle tip) is covered with a sheath. The catheter is typically slidable relative to the sheath through the lumen of the sheath. In some instances, during the disposing and/or injecting step, the distal tip (e.g., distal needle tip) of the catheter is unsheathed, e.g., by advancing the catheter distally relative to the sheath, or by retracting the sheath proximally relative to the catheter.

The sheath may be made of any suitable material, e.g., plastic, silicone, polyimide, polyurethane (PU), polyethylene (PE), polyvinylchloride (PVC), PTFE, latex, Teflon, rubber (e.g., red rubber), nylon, combinations thereof, or the like. When a sheath is employed, in some instances, the distal tip of the sheath comprises a light (e.g., a flashing light), and the method comprises using the light to visualize the tip of the sheath during the tunneling. According to some embodiments, whether or not a sheath is employed, the tip of the catheter may comprise a light (e.g., a flashing light), and the method comprises using the light to visualize the tip of the catheter during the tunneling.

As summarized above, the methods comprise disposing the distal end of the catheter (e.g., the distal needle tip) adjacent, or into, the ONH. Thus, in some instances, the distal end is disposed adjacent the ONH. When the distal end is disposed adjacent the ONH, the distal end may be disposed in the peripapillary SCS, and the therapeutic agent is injected/deposited into the peripapillary SCS. See, e.g., FIG. 2Di. In other embodiments, the disposing comprises disposing the needle tip into the ONH, and the therapeutic agent is injected into the ONH. See, e.g., FIG. 2Dii.

The methods may be employed to deliver a wide variety of therapeutic agents in various dosage forms to the ONH of the subject. In some embodiments, the therapeutic agent is injected as a liquid formulation. When the therapeutic agent is injected as a liquid formulation, in some instances, from 0.1 to 10 μL, such as from 1 to 8 μL (e.g., from 1 to 5 μL), or from 3 to 6 μL, of the therapeutic agent is injected adjacent or into the ONH.

Non-liquid dosage forms of therapeutic agents may also be employed. For example, in certain embodiments, the therapeutic agent is deposited as a solid implant that elutes the therapeutic agent. In certain embodiments, the solid implant is a polymer-based implant. Non-limiting examples of polymer-based drug-eluting implants that may be employed include polylactic-co-glycolic acid (PLGA), poly e-caprolactone (PCL), poly-L-lactic acid (PLLA), silicone, urethane, or acrylate implants. In some instances, the methods employ a mesoporous silica implant as the solid drug-eluting implant. According to some embodiments, the therapeutic agent is deposited as microparticles that elute the therapeutic agent. Non-limiting examples of such microparticles include drug-eluting microspheres, e.g., polyethylene glycol (PEG) drug-eluting microspheres.

The identity of the therapeutic agent will vary depending upon the condition of the subject to be treated. In some embodiments, the subject has an optic neuropathy. Non-limiting examples of optic neuropathies include glaucomatous optic neuropathy, ischemic optic neuropathy, autoimmune disease-associated optic neuropathy, optic disc drusen, toxic optic neuropathy, metabolic optic neuropathy, compressive optic neuropathy, Infiltrative optic neuropathy, traumatic optic neuropathy, and optic neuritis. According to some embodiments, the subject has glaucoma. In certain embodiments, the subject has non-arteritic anterior ischemic optic neuropathy (NAION).

In some instances, the therapeutic agent is a neurotrophic factor. A non-limiting example of a neurotrophic factor that may be delivered to the ONH of the subject is ciliary neurotrophic factor (CNTF).

As will be appreciated with the benefit of the present disclosure, the methods may be employed to treat an ONH-associated condition, including but not limited to, any of the optic neuropathies, glaucoma, and the like, described elsewhere herein. By “treat” or “treatment” is meant at least an amelioration of one or more symptoms associated with the condition of the subject (e.g., optic neuropathy, glaucoma, or the like), where amelioration is used in a broad sense to refer to at least a reduction in the magnitude of a parameter, e.g., symptom, associated with the condition being treated. As such, treatment also includes situations where the condition (e.g., optic neuropathy, such as glaucoma, or the like), or at least one or more symptoms associated therewith, are completely inhibited, e.g., prevented from happening, or stopped, e.g., terminated, such that the subject no longer suffers from the condition, or at least the symptoms that characterize the condition.

The therapeutic agent may be administered (e.g., in a pharmaceutical composition) in a therapeutically effective amount. By “therapeutically effective amount” is meant a dosage sufficient to produce a desired result, e.g., an amount sufficient to effect beneficial or desired therapeutic (including preventative) results, such as a reduction in a symptom of an optic neuropathy, such as glaucoma, or the like, as compared to a control. An effective amount can be administered in one or more administrations.

### Devices and Kits

Also provided by the present disclosure are devices and kits, which devices and kits find use in practicing the methods of the present disclosure.

In some embodiments, a device of the present disclosure comprises a body and a hollow microneedle extending distally from the body, where the hollow microneedle is adapted to create a sclerostomy posterior to the limbus of a human subject. Such a device further comprises a catheter with a distal needle tip, where the catheter is slidable relative to and distally through the hollow microneedle, and where the catheter is sized and adapted to be tunneled through the suprachoroidal space (SCS) from the sclerostomy to the ONH of the subject. Such a device further comprises an actuator operable to actuate the catheter relative to the hollow microneedle and drive the catheter to the ONH of the subject, and a valve assembly operable to provide a fluid coupling between a fluid source and the catheter. Features of the hollow microneedle, catheter, etc. may be as described in the Methods section above and are not reiterated herein for purposes of brevity.

In certain embodiments, the microneedle extends distally from the body at an angle of 50 to 60 degrees such that the microneedle inserts through the sclera at the angle. In some instances, the microneedle is mounted on a distal face of the device, where the microneedle extends at an angle of 50 to 60 degrees from and relative to the distal face. According to some embodiments, wherein the microneedle is mounted on a distal face of the device, wherein the microneedle extends substantially perpendicular from and relative to the distal face, and wherein the distal face comprises an angle of 50 to 60 degrees relative to the body of the device. See, e.g., FIG. 100 where the asterisk denotes such the angle of the face relative to the body of the device.

In some instances, the device comprises a reservoir operably coupled to the hollow microneedle. As used herein, “operably coupled” means that the two elements are in a functional relationship with each other. In certain embodiments, the device is adapted to inject a substance (e.g., a viscoelastic agent as described elsewhere herein) from the reservoir through the hollow microneedle into the SCS of the subject.

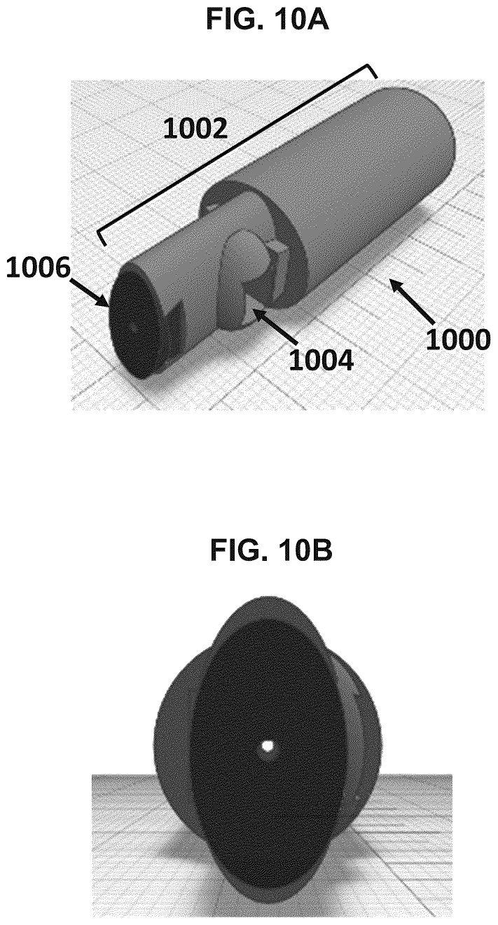

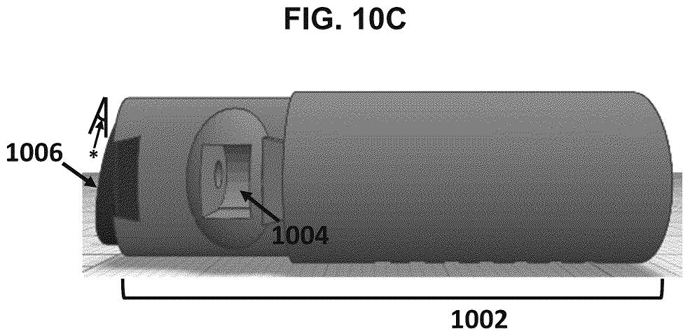

A non-limiting example of a device of the present disclosure is illustrated in FIG. 10. As shown, device 1000 comprises body 1002, reservoir 1004 (e.g., for containing a viscoelastic agent or other substance), and base plate 1006 having an angle relative to the body of the device for providing a suitable insertion angle for successful sclerostomy creation and feeding of the catheter.

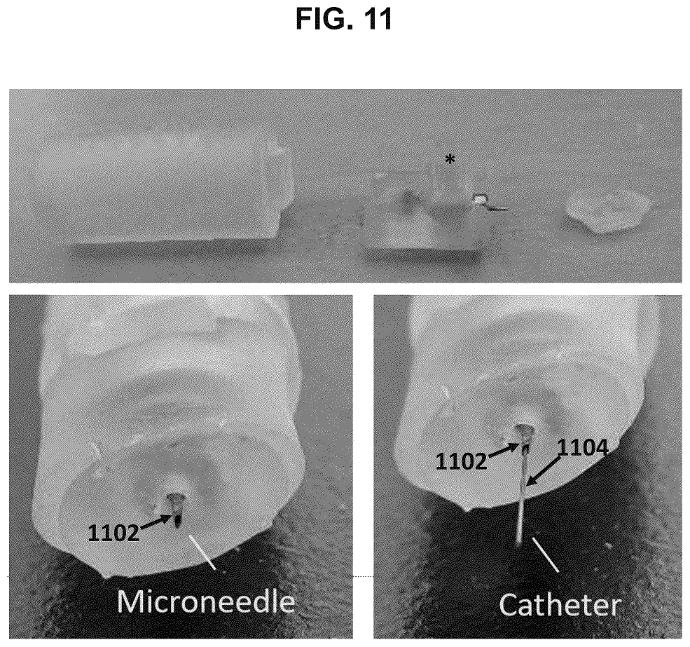

Shown in FIG. 11 is a three-dimensional printed device according to embodiments of the present disclosure. In this example, the device comprises three separate pieces including a proximal piece (left), distal piece adapted to contain a substance such as a viscoelastic agent (see asterisk), and a base plate (right). Shown on the bottom left is the device assembled and hollow microneedle 1102 mounted on the base plate. Shown on the bottom right is the assembled device with catheter 1104 extending distally from hollow microneedle 1102.

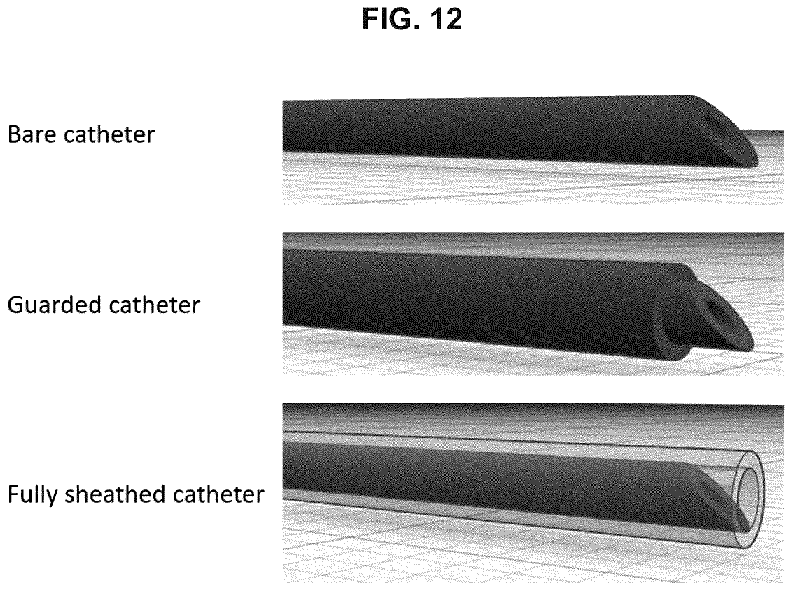

The device may implement a variety of different types of catheters, non-limiting examples of which are shown in FIG. 12. Shown on the top is a bare, unguarded catheter with a needle tip continuous with the body of the catheter. In the middle is a guarded catheter in which the body of the catheter has a larger diameter than the needle tip, thus defining the maximum distance that the needle tip can penetrate the ONH. Shown on the bottom is a sheathed catheter, which may or may not be a guarded catheter as shown in the middle.

Aspects of the present disclosure further include kits. In some instances, a kit of the present disclosure includes one or any combination of any (e.g., each) of the components of the devices of the present disclosure, and instructions for using the components for performing any of the methods of the present disclosure. In certain embodiments, a kit includes any of the devices of the present disclosure, and instructions for using the components for performing any of the methods of the present disclosure.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### EMBODIMENTS

Notwithstanding the appended claims, the present disclosure is also defined by the following embodiments:
1. A method of delivering a therapeutic agent to the optic nerve head (ONH) of a subject, the method comprising:
   creating a sclerostomy posterior to the limbus of the subject;
   feeding a catheter with a distal needle tip tangentially into the sclerostomy;
   tunneling the catheter through the suprachoroidal space (SCS) toward the ONH;
   disposing the needle tip adjacent, or into, the ONH; and
   injecting the therapeutic agent adjacent or into the ONH.
2. The method according to embodiment 1, wherein the sclerostomy is created from 1 to 5 mm posterior to the limbus.
3. The method according to embodiment 2, wherein the sclerostomy is created from 3 to 4 mm posterior to the limbus.
4. The method according to any one of embodiments 1 to 3, wherein the sclerostomy is created by inserting a hollow microneedle through the sclera.
5. The method according to embodiment 4, wherein the length of the hollow microneedle is from 500 μm to 1.5 mm.
6. The method according to embodiment 4 or embodiment 5, wherein the inner diameter of the hollow microneedle is from 200 to 400 μm.
7. The method according to any one of embodiments 4 to 6, wherein the hollow microneedle is mounted on a base plate, and wherein the angle of the microneedle is 50 to 60 degrees relative to the face of the base plate such that the microneedle inserts through the sclera at the angle.
8. The method according to any one of embodiments 4 to 7, wherein the hollow microneedle is made of metal.
9. The method according to any one of embodiments 1 to 8, wherein the outer diameter of the catheter is from 200 to 300 μm.
10. The method according to any one of embodiments 1 to 9, wherein the inner diameter of the catheter is from 100 to 200 μm.
11. The method according to any one of embodiments 1 to 10, wherein the catheter body is made of plastic.
12. The method according to any one of embodiments 1 to 11, wherein the distal needle tip is made of metal.
13. The method according to any one of embodiments 1 to 12, wherein the needle tip bevel angle is from 25 to 60 degrees.
14. The method according to any one of embodiments 4 to 13, wherein the feeding comprises passing the catheter through the lumen of the hollow microneedle.
15. The method according to any one of embodiments 4 to 14, wherein prior to the feeding, a viscoelastic agent is injected into the SCS while the hollow microneedle is still inserted through the sclera.
16. The method according to any one of embodiments 1 to 15, wherein prior to the feeding, a viscoelastic agent is injected into the suprachoroidal space (SCS).
17. The method according to embodiment 16, wherein the viscoelastic agent is a cohesive viscoelastic agent.
18. The method according to any one of embodiments 1 to 17, wherein the feeding comprises feeding the catheter tangential to the sclera at an angle of from 25 to 35 degrees.
19. The method according to any one of embodiments 1 to 18, wherein during the feeding and tunneling steps, the catheter including distal needle tip is covered with a sheath.
20. The method according to embodiment 19, wherein during the disposing and/or injecting step, the needle tip is unsheathed.
21. The method according to embodiment 19 or embodiment 20, wherein the sheath is a plastic sheath.
22. The method according to embodiment 21, wherein the distal tip of the sheath comprises a light, and wherein the method comprises using the light to visualize the tip of the sheath during the tunneling.
3. The method according to any one of embodiments 1 to 22, wherein the tip of the catheter comprises a light, and wherein the method comprises using the light to visualize the tip of the catheter during the tunneling.
24. The method according to any one of embodiments 1 to 23, wherein the disposing comprises disposing the needle tip adjacent the ONH.
25. The method according to embodiment 24, wherein the needle tip is disposed in the peripapillary SCS, and wherein the therapeutic agent is injected into the peripapillary SCS.
26. The method according to any one of embodiment 1 to 25, wherein the disposing comprises disposing the needle tip into the ONH, and wherein the therapeutic agent is injected into the ONH.
27. The method according to any one of embodiments 1 to 26, wherein the therapeutic agent is injected as a liquid formulation.
28. The method according to embodiment 27, wherein from 0.1 to 10 μL of the therapeutic agent is injected adjacent or into the ONH.
29. The method according to embodiment 28, wherein from 1 to 5 μL of the therapeutic agent is injected adjacent or into the ONH.
30. The method according to any one of embodiments 1 to 26, wherein the therapeutic agent is deposited as a solid implant that elutes the therapeutic agent.
31. The method according to embodiment 30, wherein the solid implant is a poly(lactic-co-glycolic acid) (PLGA) implant or a mesoporous silica implant.
32. The method according to any one of embodiments 1 to 26, wherein the therapeutic agent is deposited as microparticles that elute the therapeutic agent.
33. The method according to any one of embodiments 1 to 32, wherein the subject has an optic neuropathy.
34. The method according to embodiment 33, wherein the optic neuropathy is a glaucomatous optic neuropathy, ischemic optic neuropathy, autoimmune disease-associated optic neuropathy, optic disc drusen, toxic optic neuropathy, metabolic optic neuropathy, compressive optic neuropathy, Infiltrative optic neuropathy, traumatic optic neuropathy, or optic neuritis.
35. The method according to embodiment 33, wherein the subject has glaucoma.
36. The method according to embodiment 33, wherein the subject has non-arteritic anterior ischemic optic neuropathy (NAION).
37. The method according to any one of embodiments 1 to 36, wherein the therapeutic agent is a neurotrophic factor.
38. The method according to embodiment 37, wherein the neurotrophic factor is ciliary neurotrophic factor (CNTF).
39. A method of treating an ONH-associated condition in a subject in need thereof, the method comprising delivering a therapeutic agent to the ONH of the subject according to the method of any one of embodiments 1 to 38.
40. A device for delivering a therapeutic agent to the ONH of a subject, the device comprising:
   a body;
   a hollow microneedle extending distally from the body and adapted to create a sclerostomy posterior to the limbus of a human subject;
   a catheter with a distal needle tip, wherein the catheter is slidable relative to and distally through the hollow microneedle, and wherein the catheter is sized and adapted to be tunneled through the suprachoroidal space (SCS) from the sclerostomy to the ONH of the subject;
   an actuator operable to actuate the catheter relative to the hollow microneedle and drive the catheter to the ONH of the subject; and
   a valve assembly operable to provide a fluid coupling between a fluid source and the catheter.
41. The device of embodiment 40, wherein the length of the microneedle is from 500 μm to 1.5 mm.
42. The device of embodiment 40 or embodiment 41, wherein the inner diameter of the microneedle is from 200 to 400 μm.
43. The device of any one of embodiments 40 to 42, wherein the microneedle extends distally from the body at an angle of 50 to 60 degrees such that the microneedle inserts through the sclera at the angle.
44. The device of any one of embodiments 40 to 43, wherein the microneedle is mounted on a distal face of the device, and wherein the microneedle extends at an angle of 50 to 60 degrees from and relative to the distal face.
45. The device of any one of embodiments 40 to 43, wherein the microneedle is mounted on a distal face of the device, wherein the microneedle extends substantially perpendicular from and relative to the distal face, and wherein the distal face comprises an angle of 50 to 60 degrees relative to the body of the device.
46. The device of any one of embodiments 40 to 45, wherein the outer diameter of the catheter is from 200 to 300 μm.
47. The device of any one of embodiments 40 to 46, wherein the inner diameter of the catheter is from 100 to 200 μm.
48. The device of any one of embodiments 40 to 47, wherein the distal needle tip of the catheter has a bevel angle of from 25 to 60 degrees.
49. The device of any one of embodiments 40 to 48, wherein the device is adapted to feed the catheter tangential to the sclera at an angle of from 25 to 35 degrees.
50. The device of any one of embodiments 40 to 49, wherein the device comprises a reservoir operably coupled to the hollow microneedle.
51. The device of embodiment 50, wherein the device is adapted to inject a substance from the reservoir through the hollow microneedle into the SCS of the subject.
52. The device of embodiment 51, wherein the substance is a viscoelastic agent.
53. A kit comprising:
   the device of any one of embodiments 40 to 52; and
   instructions for using the device to perform the method of any one of embodiments 1 to 39.
      The following examples are offered by way of illustration and not by way of limitation.

### EXPERIMENTAL

### Example 1—SupraChoroidal-to-Optic-NErve (SCONE) Tunneling Injection

Described herein is an injection technique which may be implemented to deliver therapeutic agents to the optic nerve head (ONH) via the suprachoroidal space (SCS). An embodiment of this approach, sometimes referred to herein as SupraChoroidal-to-Optic-NErve (SCONE) tunneling injection, is schematically illustrated in FIG. 2. The data demonstrates the ability of this approach to selectively access the peripapillary SCS or ONH. The method enables reliable, safe, and efficacious targeted delivery to the ONH.

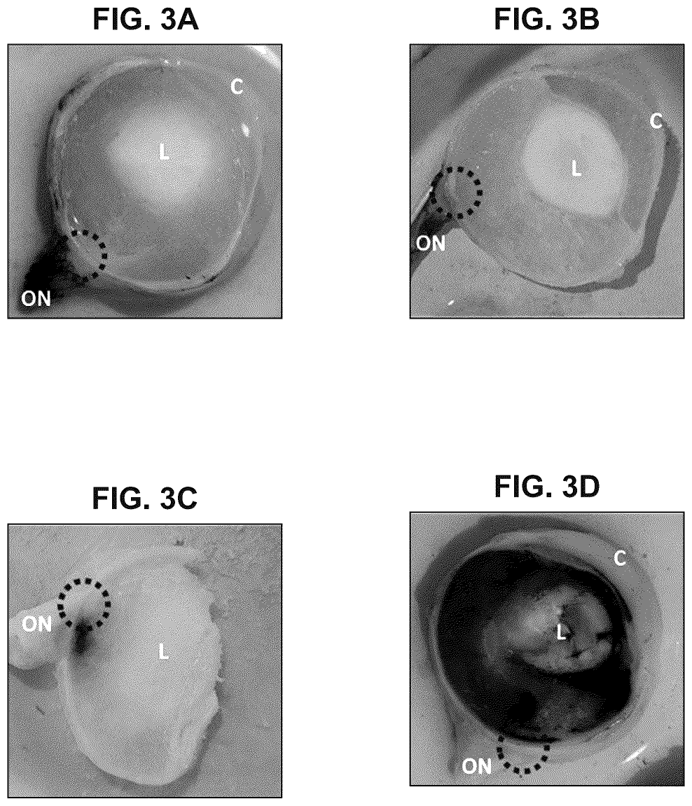

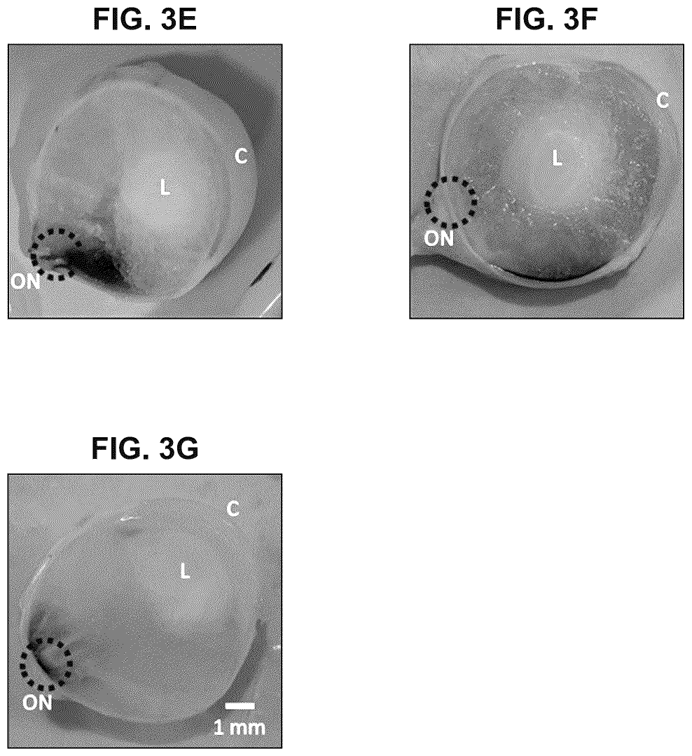

Shown in FIG. 3 are representative images of enucleated albino rabbit eyes snap frozen after injection with India ink using the following injection approaches: (A) Intraorbital optic nerve injection, (B) Intraorbital optic nerve injection with microneedle, (C) Intraorbital optic nerve injection angled toward optic nerve head, (D) Intravitreal injection, (E) Intravitreal approach to optic nerve head, (F) Suprachoroidal injection, (G) Suprachoroidal tunneling to optic nerve head (SCONE). Circle indicates 1 mm radius centered on optic nerve head. C=cornea, L=lens, ON=optic nerve stalk.

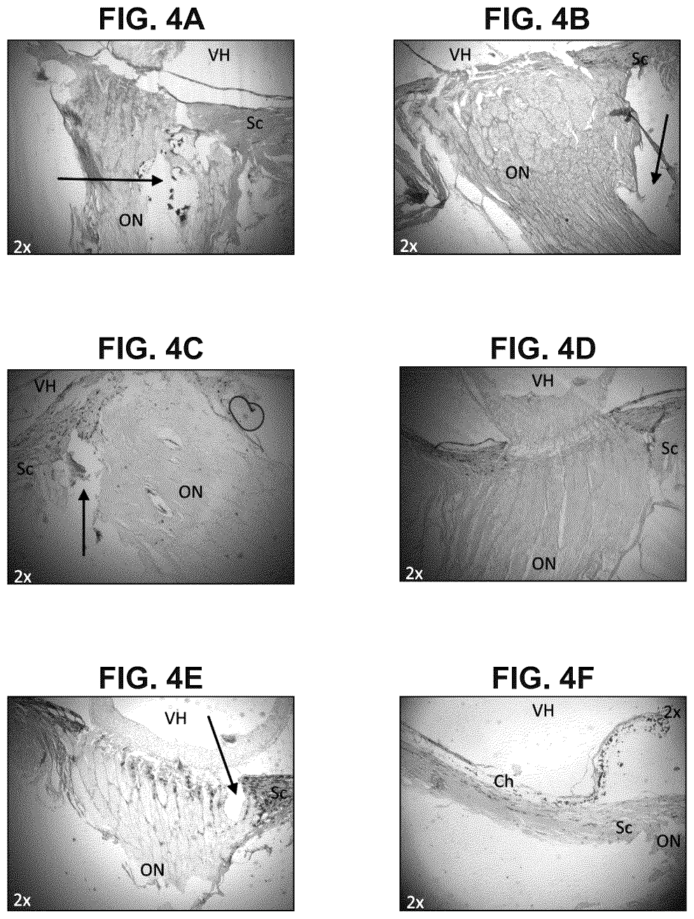

Shown in FIG. 4 are histologic photos of calcium stained with Alizarin Red delivered via (A) intraorbital optic nerve injection, (B) intraorbital optic nerve injection with a microneedle, (C) intraorbital optic nerve injection angled toward optic nerve head, (D) intravitreal injection, (E) intravitreal approach to optic nerve, and (F) suprachoroidal tunneling to the optic nerve (SCONE). Arrow indicates needle track if present on slide. VH-vitreous humor, Sc-sclera, ON-optic nerve, Ch-choroid.

Of these techniques, (A) and (B) were able to inject into the intraorbital segment of the optic nerve, but did not distribute to the ONH. Injection techniques (C) and (E) were able to deliver to the ONH but damaged the lamina cribrosa (LC). Injection technique (D) did not deliver to the ONH. Surprisingly, out of the tested injection techniques, only technique (F) (SCONE) was able to deliver to the ONH without significant damage to the lamina cribrosa.

TABLE
Summary of optic nerve delivery techniques

| | Distribution | Uninjured |
|---|---|---|
| | within ONH? | LC? |
| (A) Intraorbital optic nerve injection | N | Y |
| (B) Intraorbital optic nerve injection with a | N | Y |
| microneedle | | |
| (C) Intraorbital optic nerve injection angled | Y | N |
| toward ONH | | |
| (D) Intravitreal injection | N | Y |
| (E) Intravitreal approach to ON | Y | N |
| (F) Suprachoroidal tunneling to ON | Y | Y |

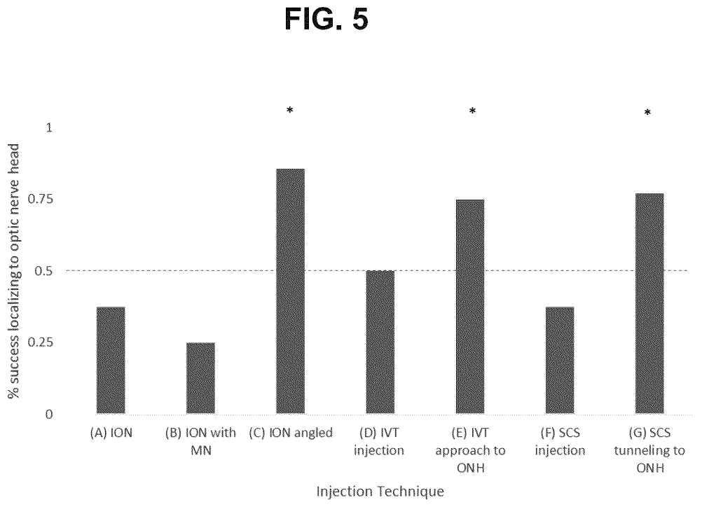

FIG. 5 provides data showing the proportion of successful localization to optic nerve head (ONH) for each injection technique. (A) Intraorbital optic nerve injection, or ION; (B) Intraorbital optic nerve injection with microneedle, or ION with MN; (C) Intraorbital optic nerve injection angled toward optic nerve head, or ION angled; (D) Intravitreal injection, or IVT; (E) Intravitreal approach to optic nerve head, or IVT approach to ONH; (F) Suprachoroidal injection, or SCS injection; (G) Suprachoroidal tunneling to optic nerve head (SCONE) * indicates significance (p<0.05) based on binomial distribution compared with 50% chance.

### Example 2—Effects of Microneedle Length and Microneedle Insertion Angle on Sclerostomy Creation

Described in this example is the assessment of microneedle length and microneedle insertion angle on sclerostomy creation.

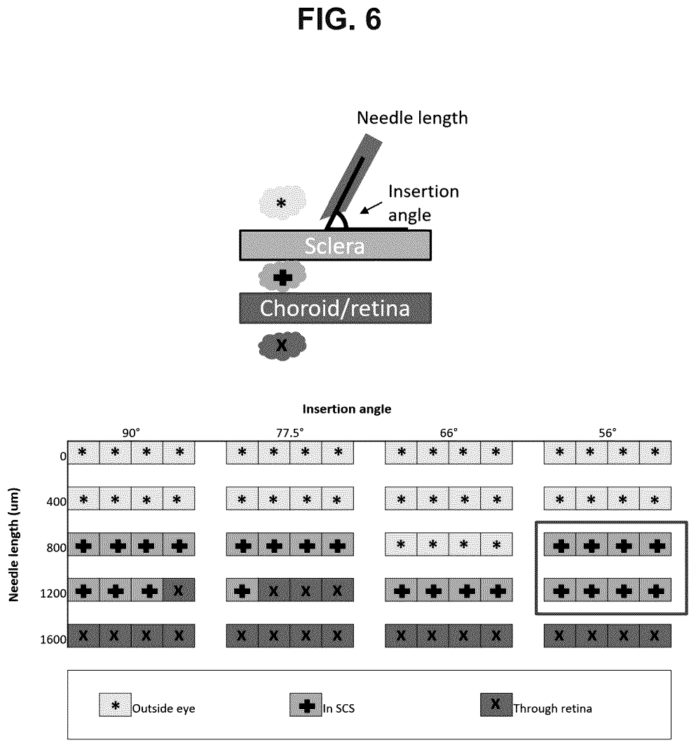

The insertion angle was varied from 90 degrees (perpendicular to sclera) to 56 degrees. The length of the microneedle was varied from 0 mm to 1.6 mm. After applying the microneedle to the sclera at each needle length and insertion angle, a catheter was extended through the lumen of the microneedle. The disposition of the catheter tip was recorded as outside the eye (microneedle had not created a successful sclerostomy), within the suprachoroidal space (microneedle had created a successful sclerostomy without penetrating through choroid and retina), and through retina (microneedle had penetrated through sclera, choroid, and retina). Five replicates were performed for each condition to determine the optimal length of microneedle and insertion angle to enter the suprachoroidal space. A microneedle length of 800 to 1200 μm and insertion angle of 56 degrees was chosen due to reliable access into the suprachoroidal space. Results are summarized in FIG. 6.

### Example 3—Successful Delivery of India Ink to the ONH Via SCONE Injection

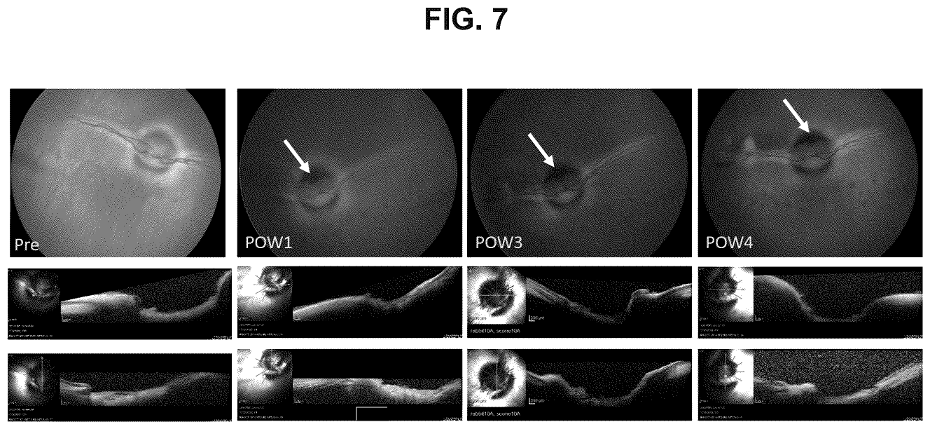

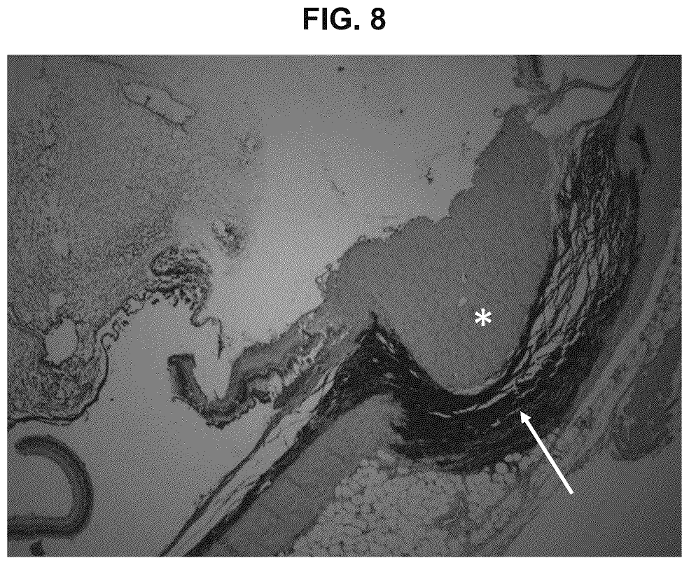

Shown in this example is successful delivery of India ink to the ONH of a rabbit eye via SCONE injection. The rabbit optic nerve head is visible pre-injection on fundus camera and optical coherence tomographic (OCT) images of the optic nerve head (FIG. 7, left). After SCONE delivery of India ink, the presence of India ink is visible at the optic nerve head on postoperative weeks 1, 3 and 4. OCT images demonstrate minimal change in ONH morphology at all time points. At 4 weeks post-SCONE injection, the animal was sacrificed and eye enucleated for histologic process. FIG. 8 shows H&E stain 4 weeks after SCONE injection, the optic nerve head is bathed with India ink.

### Example 4—Retention of Eye-Brain Connectivity Post-SCONE Injection

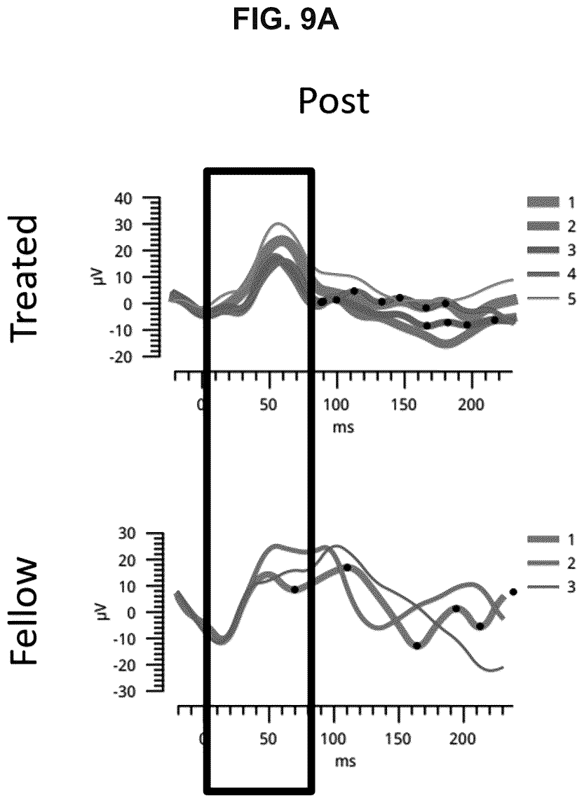

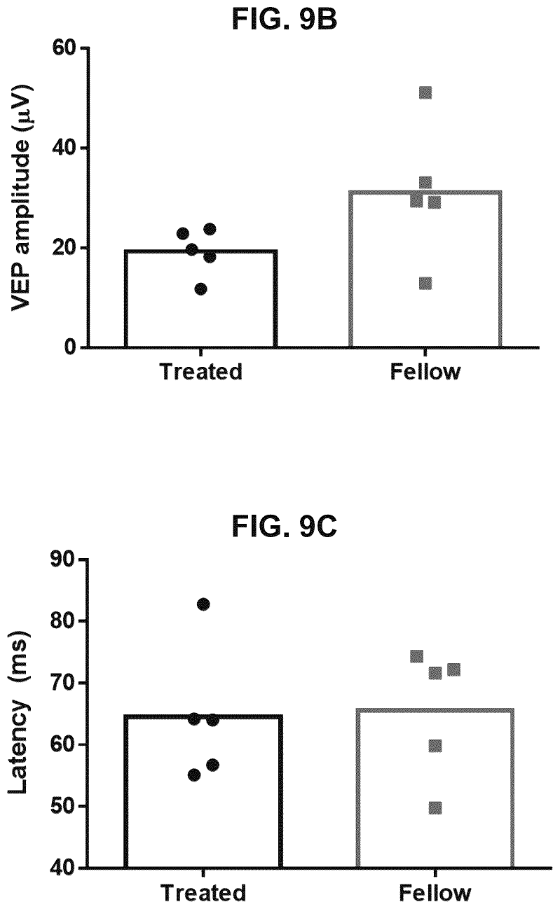

Assessed in this example was whether SCONE injection disrupts connectivity between the eye and brain. Visual evoked potentials were performed on the SCONE-treated eye and its fellow untreated eye 3 weeks after SCONE injection. Representative VEP recordings from one animal are shown in FIG. 9A. Quantification of the VEP amplitude and latency are shown in 9B. There was no significant difference between the treated and fellow eyes for either VEP amplitude or latency.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of delivering a therapeutic agent to the optic nerve head (ONH) of a subject, the method comprising:
creating a sclerostomy posterior to the limbus of the subject;
feeding a catheter with a distal needle tip tangentially into the sclerostomy;
tunneling the catheter through the suprachoroidal space (SCS) toward the ONH;
disposing the needle tip adjacent, or into, the ONH; and
injecting the therapeutic agent adjacent or into the ONH.

2. The method according to claim 1, wherein the sclerostomy is created from 1 to 5 mm posterior to the limbus.

3. The method according to claim 1, wherein the sclerostomy is created by inserting a hollow microneedle through the sclera.

4. The method according to claim 3, wherein the length of the hollow microneedle is from 500 μm to 1.5 mm.

5. The method according to claim 3, wherein the hollow microneedle insertion angle is 50 to 60 degrees.

6. The method according to claim 3, wherein the feeding comprises passing the catheter through the lumen of the hollow microneedle.

7. The method according to claim 3, wherein prior to the feeding, a viscoelastic agent is injected into the SCS while the hollow microneedle is inserted through the sclera.

8. The method according to claim 1, wherein the feeding comprises feeding the catheter tangential to the sclera at an angle of from 25 to 35 degrees.

9. The method according to claim 1, wherein during the feeding and tunneling steps, the catheter including distal needle tip is covered with a sheath, and wherein during the disposing and/or injecting step, the needle tip is unsheathed.

10. The method according to claim 1, wherein the disposing comprises disposing the needle tip in the peripapillary SCS adjacent the ONH.

11. The method according to claim 1, wherein the disposing comprises disposing the needle tip into the ONH, and wherein the therapeutic agent is injected into the ONH.

12. The method according to claim 1, wherein the therapeutic agent is injected as a liquid formulation, a solid implant that elutes the therapeutic agent, or microparticles that elute the therapeutic agent.

13. The method according to claim 1, wherein the subject has an optic neuropathy.

14. The method according to claim 13, wherein the optic neuropathy is a glaucomatous optic neuropathy, ischemic optic neuropathy, autoimmune disease-associated optic neuropathy, optic disc drusen, toxic optic neuropathy, metabolic optic neuropathy, compressive optic neuropathy, Infiltrative optic neuropathy, traumatic optic neuropathy, or optic neuritis.

15. The method according to claim 13, wherein the subject has glaucoma or non-arteritic anterior ischemic optic neuropathy (NAION).

16. The method according to claim 1, wherein the therapeutic agent is a neurotrophic factor.

17. A device for delivering a therapeutic agent to the ONH of a subject, the device comprising:
a body;
a hollow microneedle extending distally from the body and adapted to create a sclerostomy posterior to the limbus of a human subject;
a catheter with a distal needle tip, wherein the catheter is slidable relative to and distally through the hollow microneedle, and wherein the catheter is sized and adapted to be tunneled through the suprachoroidal space (SCS) from the sclerostomy to the ONH of the subject;
an actuator operable to actuate the catheter relative to the hollow microneedle and drive the catheter to the ONH of the subject; and
a valve assembly operable to provide a fluid coupling between a fluid source and the catheter.

18. The device of claim 17, wherein the length of the microneedle is from 500 μm to 1.5 mm.

19. The device of claim 17, wherein the microneedle extends distally from the body at an angle of 50 to 60 degrees such that the microneedle inserts through the sclera at the angle.

20. The device of claim 17, wherein the device comprises a reservoir operably coupled to the hollow microneedle.
